(19) United States
(12) Patent Application Publication
(10) Pub. No.: US 2008/0045933 A1
(43) Pub. Date: Feb. 21, 2008

(54) Multi-broadband pulse emitter and a method for applying an effective dermal treatment

(76) Inventor: Paul Perl, Tel-Aviv (IL)

Correspondence Address: Mark M. Friedman;Dr. Mark Friedman Ltd., c/o Discovery Dispatch, 900 Florin Way, Upper Marlboro, MD, 20772, US

(21) Appl. No.: 11/504,068

(22) Filed: Aug. 15, 2006

(51) Int. Cl. A61B 18/18; A61N 5/06

(52) U.S. Cl. 606/9; 607/88

(57) ABSTRACT

The present invention discloses a multi-broadband pulsed light emitter (MPLE) (1000) for applying an effective dermal treatment characterized by painless feature despite a high amount of applied light energy, and is also characterized by avoiding high excessive overheating of the skin layers, said MPLE is adapted to deliver energy of homogeneous and concentrated distribution (100) throughout a large focal spot (10), comprising; (a) a large broadband polychromatic source module (200), which provides a controlled pulsed light radiation for irradiating a predetermined portion of a skin to be treated (300); (b) a controller (400) adapted to select parameters selected form a group including intensity of output energy, pulse duration and number of pulses; and, (c) a cooling mechanism for simultaneously cooling both light source (250) and said treated portion of skin (300).

### FIELD OF THE INVENTION

The present invention generally relates to a multi-broadband pulsed light emitter (MPLE) for applying an effective dermal treatment and method thereof. More specifically, the present invention relates to a MPLE adapted to deliver a homogeneous and concentrated energy throughout the large focal spot size.

### BACKGROUND OF THE INVENTION

The wavelength selected for the radiation is usually determined by the absorption characteristics of the chromophore. The wavelengths typically used for treating vascular lesions are highly scattered in the skin, and only a fraction of the energy delivered to the skin surface, attains the region to be treated, limiting the efficiency of the treatment. The energy is either scattered and does not reach the treated region, or is absorbed in overlying or surrounding the skin layers, causing unwanted and dangerous heating of such tissue. The treatment is thus inefficient and the therapeutic result is relatively low.

U.S. Pat. No. 5,586,981 discloses a method provided for disrupting the targeted lesion in skin, such as is necessary in treatment of vascular or pigmented lesions. Microplasma is generated in a target region of skin, the microplasma disrupting the skin (“plasma ablation”) to enable removal of the targeted lesion. The microplasma absorbs radiation energy and expands, creating high pressure in the surrounding region that causes disruption of the targeted lesion in that region. A beam of pulsed laser radiation can be used to generate the microplasma by properly controlling the peak irradiance, the pulse duration and the focal spot size of the beam. The invention enables use of a laser having small pulse energy. A synchronized laser beam scan device can be used to scan the beam to provide a highly efficient system for rapid skin treatment.

While this approach has limited the damage to the collateral tissue by using a laser pulse, the treatment is applied with a focal spot of diameter maintained between approximately 5 micrometers and approximately 500 micrometers.

GB Pat. 2,368,020 presents an apparatus for the cosmetic treatment of a skin condition which comprises means for delivering illuminating radiation to a target skin zone or structure.

Moreover, another important disadvantage of the existing treatment is the patient pain management. The amount of energy which can be applied to the treated skin region is limited by the patient pain. A local anesthetic is usually applied, and a high amount of energy is applied to obtain an efficient treatment.

Performing such treatments with a more powerful emitter or utilizing a larger number of more powerful emitters is more expensive, increases the size of the device and increases the heat management problems.

Moreover, stretch marks or striae distensae are a very common problem for which treatment remains a challenge. In the early stages, striae appears pink to red (striae rubra), which over time becomes atrophic and attains a white color (striae alba). On histopathology, striae distensae are very similar to scars with a thin, flattened epidermis, attenuation of the rate ridges, fraying and separation with orientation of collagen bundles in a horizontal plane, dilatation of blood vessels, and abundant clumped elastic fibers. The causes of stretch marks are numerous, including mechanical stress, such as weight changes and weight lifting, corticosteroid therapy, Cushing's syndrome, infections, and hormonal factors such as puberty and pregnancy.

Furthermore, anti aging attitudes of western societies are also creating challenges to dermatologists, plastic surgeons and physicians around the world. The use of ablative lasers is limited due to prolonged healing time and the risk of complications. A new trend in skin surgery has led to the development of non ablative lasers and light sources for the treatment of UV-damaged, scarred and injured skin. Various treatment options for rejuvenation of photo damaged skin have been developed in the past. Some of them lost popularity because of the risk of undesired side effects and prolonged periods of recovery. Aesthetically oriented patients seek for less invasive methods a variety of new light sources has been encountered. Most of these devices stimulate fibroblasts and create new collagen production with more or less side effects. Still, assessment of the resulting improvement in the appearance of photo damaged skin is a delicate issue.

Moreover, acne is a common disorder that may result in permanent scars. Recently a simple and universally applicable classification system has been proposed for acne scars, which have been divided into 3 basic types: icepick scars, rolling scars and boxcar scars. Icepick scars are narrow, deep, sharply emarginated epithelial tracts that extend vertically to the deep dermis or subcutaneous tissue. Their depth is below that reached with conventional skin resurfacing options and complete recovery is usually impossible. Rolling scars occur from dermal tethering of skin. Abnormal fibrous anchoring of the dermis to the subcutis leads to superficial shadowing and a rolling appearance to the overlying skin. Although they tend to be shallow, the sub dermal tether precludes treatment from the surface above. Correction of the sub dermal component is essential for treatment success. Boxcar scars are round or oval depressions with sharply demarcated vertical edges. They are clinically wider at the surface than icepick scars; they may be shallow (e.g., 0.1 to 0.5 mm) or deep (e.g., greater than 0.5 mm). Shallow boxcar scars are within the dermal reach of skin resurfacing treatments, but deeper boxcar scars do not improve in absence of a full thickness treatment technique.

A variety of approaches are available for revision of each of the 3 scar types. Most of them are surgical or invasive procedures that may have a long and unacceptable downtime for some patients. Moreover all resurfacing treatments usually require a sub surfacing “filling” to correct the depression. One of the newest trends of scar treatment has been the development of no ablative light or laser systems that determine a collagen remodeling effect that can achieve a consistent improvement of depressed acne scars.

None of these prior art references disclose a system that can deliver a high dose of light energy through large local spot size, with better penetration capacity, for more efficient treatment with no pain involved.

Furthermore, none of these prior art references disclose a non-invasive safe, painless and effective treatment for patients who desire rejuvenation of photo damaged skin.

Under all these reasons, and for such treatments, a more concentrated and homogeneous light energy source is required to overcome this low efficiency and pain related due to high excessive overheating of the skin layers.

### SUMMARY OF THE INVENTION

It is thus one object of the present invention to provide an efficient multi-broadband pulsed light emitter (MPLE) for applying an effective dermal treatment.

It is in the scope of the present invention wherein said effective dermal treatment is characterized by painless feature despite a high amount of applied light energy, and is also characterized by avoiding high excessive overheating of the skin layers

It is also in the scope of the present invention wherein said MPLE is adapted to deliver energy of homogeneous and concentrated distribution throughout a large focal spot.

Said MPLE comprises a large broadband polychromatic source module, which provides a controlled pulsed light radiation for irradiating a predetermined portion of a skin to be treated; a controller adapted to select parameters selected form a group including intensity of output energy, pulse duration and number of pulses; and, a cooling mechanism for simultaneously cooling both light source and said treated portion of skin.

It is also in the scope of the present invention wherein the broadband polychromatic source module comprising: a light source, such that a flash lamp or a gas discharge arc lamp, comprising an anode and a cathode housed in a tube; said light source is characterized by a stronger discharge in the middle of said tube than near said cathode and anode, such that near each extremity scattered photons are emitted inducing non-homogeneous and inefficient energy; a main reflector, positioned in parallel to the axis of said light source, reflecting said light backwards towards said light source; at least two reflectors each of which is located on a side of said light source, reflecting said non-homogeneous and inefficient light emitted near said anode and cathode back in the direction of the main reflector; wherein said MPLE concentrates the bulk of the energy in the middle of the lamp and to a geometric irradiation plane perpendicular to said portion of skin to be treated such that an homogeneous and concentrated energy is emitted.

It is further in the scope of the present invention wherein said controlled pulsed light is applied in differently chopped modes into series of mini pulses light at durations of about 500 ms to 3000 ms, with about 10 ms to 200 ms interval between said pulses.

It is further in the scope of the present invention wherein said light source (250) is a glass xenon flash lamp.

It is further in the scope of the present invention wherein said light source emission is in the range of about 600 nm to 1,850 nm.

It is further in the scope of the present invention wherein the length of said large focal spot size lies in the range of about 30 mm to about 50 mm.

It is further in the scope of the present invention wherein the width of said large focal spot size lies in the range of about 10 mm to about 20 mm.

It is further in the scope of the present invention wherein said cooling mechanism combines; an internal air-cooled light source, adapted to controllably emit light towards said treated skin; and, a liquid-cooled skin contact means adapted to provide a painless dermal treatment and to prevent overheating of said treated skin by said light.

It is further in the scope of the present invention wherein said output energy lies in the range of about 20 J/cm² to about 65 J/cm².

It is further in the scope of the present invention wherein said normalization of the output energy over the large focal spot size surface lies in the range of about 36 J to about 390 J.

It is still in the scope of the present invention to provide a method for delivering energy homogeneously and in a concentrated manner throughout the large focal spot size comprising the steps of emitting a controlled pulsed polychromatic light radiation towards a predetermined region of a skin to be treated; cooling simultaneously both said light radiation and said treated skin; controlling the intensity of said output energy, the pulse duration and the number of pulses; such that an efficient dermal treatment characterized by painless feature despite a high amount of applied light energy, and by avoiding high excessive overheating of the skin layers is obtained.

It is also in the scope of the present invention wherein the method for building up a thermal effect, comprises applying at least two sets of chopped light pulses having time intervals of about 2 to 5 sec.

It is further in the scope of the present invention wherein the step of controlling said intensity of said output energy is applied according to the characteristics features of skin to be treated (300), to the depth within the skin at which treatment is desired, and to the absorption of said energy in the desired predetermined portion of skin.

It is further in the scope of the present invention wherein the method is adapted to the treatments of stretch marks, acne, acne scars and vascular lesions and collagen remodeling.

It is further in the scope of the present invention wherein the method is especially adapted for collagen remodeling, especially adapted for treating patients with atrophic facial scars or fine wrinkles.

It is lastly in the scope of the present invention wherein a non-invasive method, especially adapted for skin tightening, comprising step or steps of deep dermal heating and fibroblast stimulating is provided.

While the invention is susceptible to various modifications and alternative forms, specific embodiments thereof have been shown by way of example in the drawings and will herein be described in detail. It should be understood, however, that it is not intended to limit the invention to the particular forms disclosed, but on the contrary, the intention is to cover all modifications, equivalents, and alternatives falling within the spirit and scope of the invention as defined by the appended claims.

### BRIEF DESCRIPTION OF THE FIGURES

In order to understand the invention and to see how it may be implemented in practice, and by way of non-limiting example only, with reference to the accompanying drawing, in which

FIG. 1 schematically presents a simplified and out of scale cross view diagram of the large broadband polychromatic source module 200 according to one embodiment of the present invention;

FIG. 2 represents a simplified and out of scale cross view diagram of the emitter 1000;

FIGS. 3 represents two stacked burst pulses for building up the thermal effect with low pain level as a function of the temperature according to one embodiment of the present invention; and,

FIG. 4 represents a not in scale scheme illustrating the same.

### DETAILED DESCRIPTION OF PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a multi-broadband pulsed light emitter (MPLE) for applying an effective dermal treatment adapted to deliver a homogeneous and concentrated energy throughout the large focal spot size without the need of any anesthetics.

The term ‘homogeneous energy distribution’ refers hereinafter to a homogeneous distribution of the effective radiation energy of the source over the longitudinal direction of the irradiation plane, to avoid excessive heating or thermal damage to the surrounding tissue.

The term ‘homogeneous energy distribution’ also refers to the stable and constant features of the energy.

The term ‘concentrated energy distribution’ refers hereinafter to the high kinetic thermal energy concentrated to a target area, to obtain a more efficient treatment.

The term ‘efficient treatment’ refers hereinafter to the painless feature of the treatment despite the high amount of energy applied.

The term ‘building up a thermal effect’ refers hereinafter to the slower pace of elevating desired temperature to the dermis and hypodermis while keeping minimal pain levels in the epidermis.

The term ‘about’ refers hereinafter to a tolerance of ±20% of the defined measure.

The wavelengths typically used for treating vascular lesions are about 650 nm to about 1800 nm. These wavelengths are highly scattered in the skin, and only a fraction of homogeneous light needed is delivered to the skin, attains the region to be treated. The energy is either scattered and does not reach the treated region, or some is absorbed in overlying layers, and, or surrounding the skin layers, causing unwanted and dangerous heating of such tissue.

The present invention discloses a pulsed light emitter that delivers a homogeneous and concentrated energy throughout the large focal spot size. A beam distribution system that provides a homogeneous energy density distribution is of advantage, since such a system permits safe application of the light source in the effective therapeutic range. Conventional laser scanners for medical use have been reported to produce grossly inhomogeneous energy density distributions, and therefore inhomogeneous deposition of heat in the tissue. Hot spots are avoided by a particularly homogeneous energy distribution across the entire beam spot.

The homogeneous and concentrated features of the energy permit to apply the treatment on a target area of a patient's skin. Due to the targeting, the adjacent tissue is not overheated, making the treatment efficient and painless.

Moreover, another important advantage of the existing treatment is the patient pain management. The cooled glass provides a minimum pain with a maximum concentrated energy. The broadband emitter emits light via skin contact cooling.

The emitter of the present invention is based on the improvement of the capacity to deliver more photons to the dermis with minimal heat effect to the epidermis, wherein treatments for Scars, Acne, Stretch Marks, and all other applications resulting from the fibroblast effect of this homogeneous light emitter with minimal pain to the epidermis.

The optimal characteristics of the emitter are as follows: the emitter has a broadband wavelength from about 650 nm up to about 1800 nm, large spot size of about 15×40 mm, the pulse widths are of about 3 ms to about 6 ms, with about 20 ms interval between pulses at about 0.5 Hz. The maximum energy that can be delivered is about 65 J/cm2. The penetration depth lies in the range of about 2 mm to about 6 mm.

The MPLE offers clinical improvement in collagen remodeling for patients with atrophic facial scars and fine wrinkles. The visible and the IR spectra of light used by this new flash lamp achieve optimal results. Absorption, that is converted into heat, creating inflammatory response in the dermis, followed by a process of tissue repair, will boost collagen formation. The ability to progressively and slowly building up heat up to about 65° C. while keeping the epidermis protected with a cooling mechanism system, will have as consequence effective skin rejuvenation with good cosmetic results at a low rate of side effects and complications.

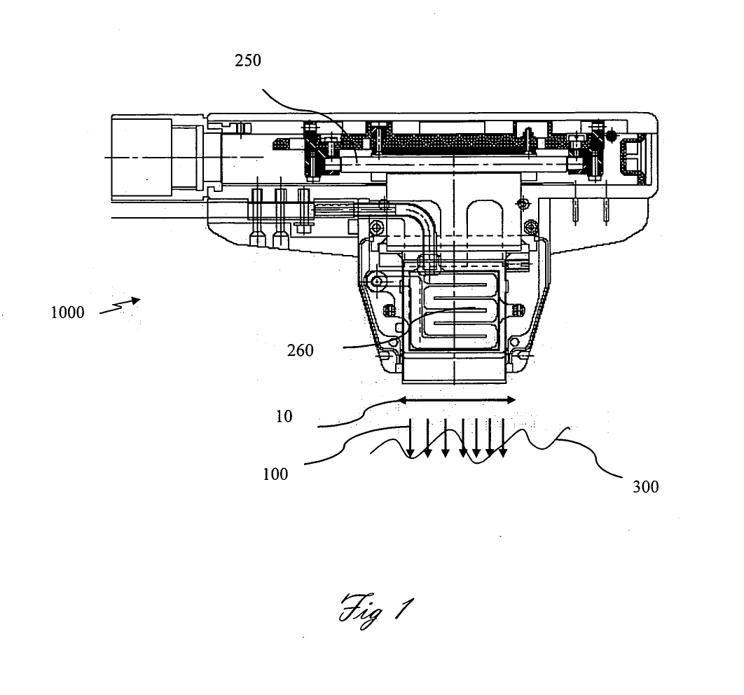

Referring now to the drawings, FIG. 1 is a schematic, simplified and out of scale cross view diagram of the emitter 1000. Optical energy 100 from a light source 250 passes a cooling mechanism before reaching the predetermined region of a skin to be treated 300. Energy source 250 may be any suitable optical energy source able to produce electromagnetic radiation such as near infrared or visible light radiation a wavelength of the range of about 600 nm to about 1850 nm. Energy source 250 may be any suitable flash lamp or gas discharge arc lamp such as the quartz xenon flash lamp model G5109, commercially available for example from The Electronic Goldmine, US. The intensity of the energy can be selectively chosen, as a function of the skin to be treated 300, of the depth within the skin at which treatment is desired, and of the absorption of that energy in the desired predetermined region. Cooling mechanism may have any suitable configuration, for example, it may combine an air-cooled light source and a liquid-cooled skin contact means.

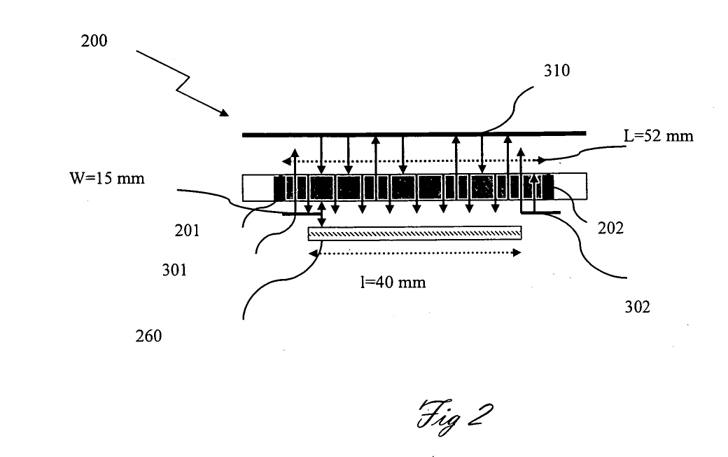

Reference is now made to FIG. 2, illustrating a schematic, simplified and out of scale cross view diagram of the emitter 1000. The space of xenon gas in the quartz between the cathode 202 and the anode (201) is in this example about 52 mm.

The discharge in the middle of the quartz tube is stronger than near the cathode 202 or the anode 201, therefore reflectors 301, 302 located about 6mm on each side of the quartz tube sent back this inefficient and non-homogeneous light emitted near said anode 201 and cathode 202, resulting in emission only in the middle 40 mm from the total 52 mm of arc.

The module comprises a main reflector 310 positioned in the axial direction of the light source sending light back in the direction of the light source. The 6 mm on each side of the anode 201 and the cathode 202 are blocked, reflecting back the scattered photons produced near each extremity to main reflector 310. The most part of the pulsed light is transmitted perpendicularly to the skin. The cooled glass spot size is hence about 40 mm, producing a homogeneous and concentrated light energy throughout the large focal spot size.

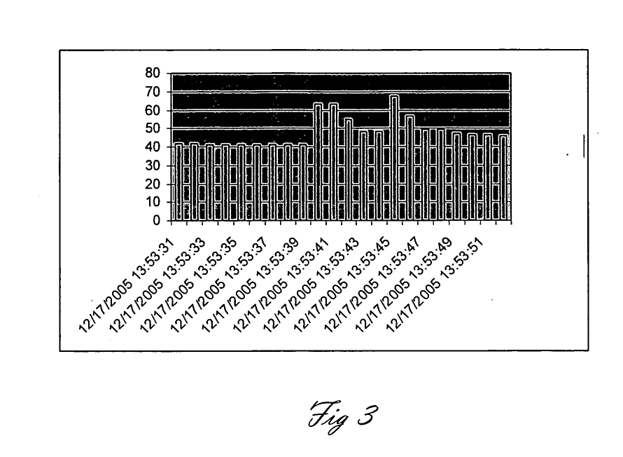

Reference is now made to FIG. 3, showing two stacked burst pulses for building up the thermal effect with no/low pain level as a function of the temperature. The treatment illustrated in FIG. 3 is performed with output energy of 28 J/cm².

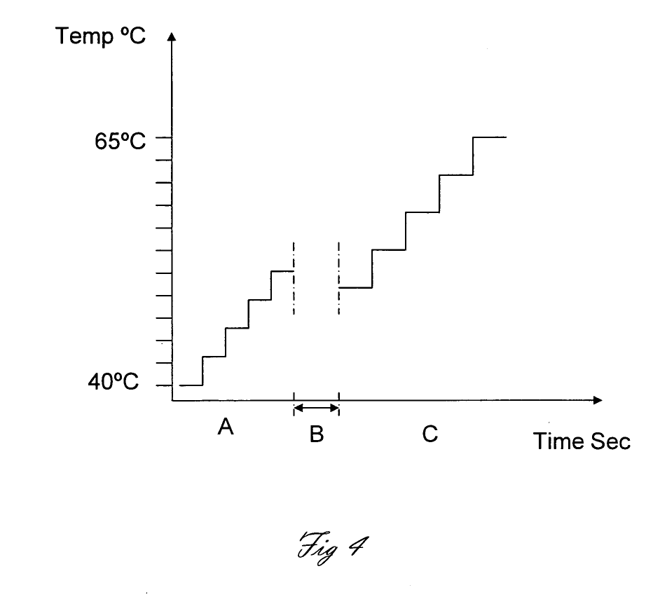

Reference is now made to FIG. 4, illustrating the application of a first dose of burst pulses (Zone A), the time interval of about 2 to 5 sec (Zone B) while a thermo cooling of the tissue is provided, and the application of a second dose of burst pulses, for slower pace of elevating desired temperature to the dermis and hypodermis while keeping minimal pain levels in the epidermis.

### EXAMPLES

Various examples were carried out to prove the embodiments claimed in the present invention. Some of these experiments are referred hereinafter. The examples describe the manner and process of the present invention and set forth the best mode contemplated by the inventors for carrying out the invention, but are not to be construed as limiting the invention.

### Example 1

The treatment of stretch marks is performed in patients with all skin types. For stretch marks treatment, the infrared light lies in the range from 800 nm to 1,800 nm, the energy density used is 31 J/cm². The light pulses may be applied in differently chopped modes at durations of 500 ms to 3.000 ms in total. All treatments are applied via single handpiece with a spot size of 6 cm² (40×15 mm) with no need of filters. An aggressive active contact skin cooling at +5° C. is activated to avoid any epidermal injury being in any skin photo types. The particular burst mode, which can be modified by a simple operation on the software, chops the pulse into a series of mini pulses. Burst pulse widths are of 3 ms to 6 ms, with 20 ms interval between pulses at 0.5 Hz.

On clinical examination and standardized photography an improvement in width from 0 to 30% is observed. Three-dimensional in vivo optical skin imaging assessed anisotropy of micro relief before and at the fourth session showed improved in depth and in micro relief from 25 to 50%. Treatment satisfaction was graded good and excellent in 40% of patients with all SPT. Any side effects were noticed

The results confirm on white or red stretch marks that the MPLE improves skin texture without any side effects or overheating. Pain in Epidermis level is low due to aggressive and active skin cooling via the hand piece. This pulse light technology is the first system to give objective results in white or red stretch marks.

### Example 2

An MPLE (1000) according to one embodiment of the present invention was used as non-invasive skin tightening protocol in periorbital areas. The infrared light lies in the range from 800 nm to 1,800 nm. The energy densities used lies in the range of 21 J/cm² to 45 J/cm², in most cases 28 J/cm² was used without local anesthesia. The light pulses may be applied in differently chopped modes at durations of 500 ms to 3.000 ms. All treatments are applied via single handpiece with a spot size of 6 cm² (40×15 mm) with no change of filters needed. To avoid epidermal injury skin contact cooling is integrated in the hand piece, cooling the skin surface down to (−) 5° C. to +5° C. is activated to avoid any epidermal injury on all skin types. The system of the present invention is able to effectively heat up dermal layers up to 65° C. while keeping the epidermis protected with an adapted cooling system. The treatment was done with a train of pulses of 300 ms in total, with fluencies of around 30 J/cm². Pulses were stacked at two times per treated spot area on the face except on bony areas where only one pulse was delivered without any type of anesthesia. Thus, creating a sub-threshold light induced injury to the dermis and/or dermal vessels leads to a wound repair response with fibroblast stimulation resulting in a skin tightening effect due to new collagen formation at a low rate of side effects and no complications.

The MPLE applied to the skin surface in chopped pulses is able to improve the clinical appearance of photo damaged skin. Three-dimensional in vivo optical skin imaging provided quantitative assessment of surface topography and periorbital wrinkles before and after three treatment sessions. The average improvement of wrinkle depth comparing before and after measurements was 18.0%, the average improvement of wrinkle width was 13.2%.

### Example 3

To evaluate the efficacy on collagen remodeling, the subdermal heating generated by the aforesaid MPLE was verified. Thermocouple probes are introduced at a controlled depth in the hypodermis by thermocouple needles. At the surface of the skin, above the probe, the MPLE is activated at a high fluency of 28 J/cm² with a train of pulses of 500 ms. An immediate response is observed between 3000 and 7000 μm. A thermal peak at 60° C. during one or two seconds in the subdermis is also observed, and the maximum pain level was 4/10 and never required additional topical anesthesia.

1. A multi-broadband pulsed light emitter (MPLE) (1000) for applying an effective dermal treatment characterized by painless feature despite a high amount of applied light energy, and is also characterized by avoiding high excessive overheating of the skin layers, said MPLE is adapted to deliver energy of homogeneous and concentrated distribution (100) throughout a large focal spot (10), comprising;
a. a large broadband polychromatic source module (200), which provides a controlled pulsed light radiation for irradiating a predetermined portion of a skin to be treated (300);
b. a controller (400) adapted to select parameters selected form a group including intensity of output energy, pulse duration and number of pulses; and,
c. a cooling mechanism for simultaneously cooling both light source (250) and said treated portion of skin (300).

2. The MPLE according to claim 1, wherein the broadband polychromatic source module (200) comprising:
a. a light source (250), such that a flash lamp or a gas discharge arc lamp, comprising an anode (201) and a cathode (202) housed in a tube; said light source is characterized by a stronger discharge in the middle of said tube than near said cathode and anode, such that near each extremity scattered photons are emitted inducing non-homogeneous and inefficient energy;
b. a main reflector (310), positioned in parallel to the axis of said light source, reflecting said light backwards towards said light source;
c. at least two reflectors (301, 302) each of which is located on a side of said light source, reflecting said non-homogeneous and inefficient light emitted near said anode (201) and cathode (202) back in the direction of the main reflector;
wherein said MPLE concentrates the bulk of the energy in the middle of the lamp and to a geometric irradiation plane perpendicular to said portion of skin (300) to be treated such that a homogeneous and concentrated energy is emitted.

3. The MPLE according to claim 1, wherein said controlled pulsed light is applied in differently chopped modes into series of mini pulses light at durations of about 500 ms to 3000 ms, with about 10 ms to 200 ms interval between said pulses.

4. The MPLE according to claim 1, wherein said light source (250) is a glass xenon flash lamp.

5. The MPLE according to claim 1, wherein said light source emission is in the range of about 600 nm to 1,850 nm.

6. The MPLE according to claim 1, wherein the length of said large focal spot size (10) lies in the range of about 30 mm to about 50 mm.

7. The MPLE according to claim 1, wherein the width of said large focal spot size (10) lies in the range of about 10 mm to about 20 mm.

8. The MPLE according to claim 1, wherein said cooling mechanism combines;
a. an internal air-cooled light source (250), adapted to controllably emit light towards said treated skin (300); and,
b. a liquid-cooled skin contact means (260) adapted to provide a painless dermal treatment and to prevent overheating of said treated skin by said light.

9. The MPLE according to claim 1, wherein said output energy lies in the range of about 20 J/cm² to about 65 J/cm².

10. The MPLE according to claim 1, wherein said normalization of the output energy over the large focal spot size surface lies in the range of about 36 J to about 390 J.

11. A method for delivering energy homogeneously and in a concentrated manner throughout the large focal spot size (10) comprising:
a. emitting a controlled pulsed polychromatic light radiation towards a predetermined region of a skin to be treated (300);
b. cooling simultaneously both said light radiation and said treated skin (300);
c. controlling the intensity of said output energy, the pulse duration and the number of pulses; such that an efficient dermal treatment characterized by painless feature despite a high amount of applied light energy, and by avoiding high excessive overheating of the skin layers is obtained.

12. The method according to claim 11, for building up a thermal effect, comprising; applying at least two sets of chopped light pulses having time intervals of about 2 to 5 sec.

13. The method according to claim 11, comprising the step of controlling said intensity of said output energy according to the characteristics features of skin to be treated (300), to the depth within the skin at which treatment is desired, and to the absorption of said energy in the desired predetermined portion of skin.

14. The method according to claim 11, adapted to the treatments of stretch marks, acne, acne scars and vascular lesions and collagen remodeling.

15. The method according to claim 14, especially adapted for collagen remodeling, especially adapted for treating patients with atrophic facial scars or fine wrinkles.

16. A non-invasive method according to claim 11, especially adapted for skin tightening, comprising step or steps of deep dermal heating and fibroblast stimulating.
